(19) United States
(12) Patent Application Publication
(10) Pub. No.: US 2024/0293128 A1
(43) Pub. Date: Sep. 5, 2024

(54) TEMPORARY BLOOD VESSEL OCCLUSION DEVICE AND METHOD

(71) Applicants: Patrick A. Harty, Omaha, NE (US); Christopher J. Laing, Sacramento, CA (US)

(72) Inventors: Patrick A. Harty, Omaha, NE (US); Christopher J. Laing, Sacramento, CA (US)

(21) Appl. No.: 18/434,072

(22) Filed: Feb. 6, 2024

Related U.S. Application Data
(60) Provisional application No. 63/443,708, filed on Feb. 6, 2023.

(51) Int. Cl. A61B 17/12; A61B 17/00

(52) U.S. Cl. CPC A61B 17/12186; A61B 17/1204; A61B 17/12109; A61B 2017/00022; A61B 2017/1205

(57) ABSTRACT

A temporary vessel occlusion system for controlling major and/or traumatic internal bleeding in patients. The temporary vessel occlusion system has location determining capabilities used to quickly deploy and locate a catheter in a vessel for the delivery of a biodegradable occlusion agent that is able to temporarily stop the bleeding or flow of a fluid out of or within the vessel.

### TECHNICAL FIELD

This disclosure relates to blood vessel occlusion devices. In particular, this disclosure relates to a device and method for temporary occluding a blood vessel, such as the abdominal aorta.

### BACKGROUND

Abdominal or visceral bleeding is often a critical, but treatable injury with the correct tools and equipment. Some contemporary means of treating or temporizing emergency thoracic or abdominal hemorrhage include resuscitative endovascular balloon occlusion of the aorta (REBOA), fluoroscopically guided transcatheter embolization, and laparotomy. However, sometimes these injuries occur in places where these advanced means of treatment are not available. Injuries may occur in active military zones where medical treatment options are limited, or even in civilian centers, where highly trained personnel or necessary equipment are not available, or there is not time to treat an injury with standard equipment and personnel.

Current methods of managing torso or abdominal hemorrhage includes preforming an emergency thoracotomy or laparotomy, both of which are major surgical procedures that involve a large opening of the chest or abdominal wall to expose the aorta or other major vessels. Such a procedure can only be performed with specialized surgical equipment in a controlled environment by a highly trained medical professional. Another method of managing torso hemorrhage includes placing a balloon occlusion device within a blood vessel. For example, a balloon occlusion device may be placed within the thoracic aorta. This procedure requires a surgical interventional team using fluoroscopy or x-ray to place a balloon catheter within the vessel to deploy a balloon. Again, this procedure can only be performed with specialized surgical equipment in a controlled environment by a highly trained medical professional.

Thus, the requirement of x-ray or fluoroscopy to use currently available balloon occlusion systems restricts performance of this procedure to fixed highly specialized operating rooms with fluoroscopy capabilities or fixed imaging suites, both of which are typically not available in field trauma or emergency settings, or lower level rural and suburban emergency care hospitals. Further, these options are not available where the fluoroscopy or x-ray machines are in disrepair. In addition, in emergency, intensive care, or surgical environments, fluoroscopy is often not readily available in the environments in which the patients are positioned, e.g., an intensive care unit (ICU) bed or operating room (OR) table, are not specifically made for imaging, thereby impeding the use of fluoroscopy.

Despite the potentially life-saving nature of balloon occlusion devices in the setting of massive torso and/or pelvic hemorrhage, current approaches for the placement of these devices require continued monitoring of the occlusion balloon catheter for deflation or other compromise in addition to a secondary operation, wherein the balloon is removed. Without removal, these devices are theoretically permanent, so they must be manually removed by a medical professional.

The present disclosure addresses the need for improvements in vessel occlusion devices and methods for the treatment of acute hemorrhagic injury.

### SUMMARY

In general terms, this disclosure is related to a catheter device and system for injecting a temporary embolic agent into a vessel (e.g., blood vessel, urethra, or any other passageway) without requiring any imaging (e.g., fluoroscopy, ultrasound, or x-ray machinery).

In a first aspect, a temporary vessel occlusion device and system is described. The occlusion device comprises a guidewire, catheter, and a temporary embolic agent. The guidewire includes at least one communication or location sensor disposed or positioned at its distal end and is capable of communicating with an external receiver that is able to display a location of the guidewire. The catheter includes at least one lumen and is in fluid communication with at least one access port. The catheter is configured to be inserted alongside or over at least a portion of the guidewire, and to deploy the temporary embolic agent into the vessel of a patient.

In another aspect, a kit for temporary vessel occlusion is described. The kit includes an insertion assisting device, a guidewire, a catheter, and a temporary embolic agent. The insertion assisting device includes at least one communication receiver. The guidewire includes at least one communication or location sensor located at its distal end. The catheter includes at least one lumen in fluid communication with at least one access port. The communication receiver is capable of communicating with the location sensor to determine the location of the guidewire within the vessel. The catheter is configured to be inserted alongside or over a guidewire and to deploy the temporary embolic agent into the vessel.

In yet another aspect, a method of providing a temporary occluding to a vessel is described. The method includes inserting a guidewire having at least one communication or location sensor into a vessel. The at least one location sensor communicates with an external communication receiver that is used to determine and display a final location of a distal end of the guidewire. A catheter is inserted alongside or over the guidewire into the vessel. A temporary embolic agent is deployed within the vessel. The guidewire and catheter are then removed from the vessel.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description. Embodiments of the present disclosure will hereinafter be described in conjunction with the appended drawings, wherein like numerals denote like elements.

FIG. 1 illustrates an example anatomical representation of the major blood vessels in the human abdomen.

FIG. 2 illustrates an example embodiment of a catheter device for supplying a temporary embolic agent.

FIGS. 3a-3c illustrate example embolic agent devices.

FIG. 4 illustrates an example delivery device.

FIG. 5 illustrates a different example delivery device.

FIG. 6 illustrates another example delivery device.

FIG. 7 illustrates a delivery device handle.

FIG. 8 illustrates an example embodiment of an insertion template.

FIG. 9 illustrates an example embodiment of insertion of the catheter device with a doppler.

FIG. 10 illustrates a sensor pad located on an operating room table.

FIG. 11 illustrates an example of a catheter device within a blood vessel.

FIG. 12 illustrates an example blood vessel with a temporary embolic agent injected therein.

FIG. 13 illustrates an example method of inserting a catheter device.

FIG. 14 illustrates an alternative example method of inserting a catheter device.

FIG. 15 illustrates an example computing system useful as part of a guidance system.

Corresponding reference characters indicate corresponding parts throughout the several views. The exemplifications set out herein illustrate an embodiment of the invention, and such exemplifications are not to be construed as limiting the scope of the invention in any manner.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical devices, systems, and methods. Those of ordinary skill may recognize that other elements and/or operations may be desirable and/or necessary to implement the devices, systems, and methods described herein. Because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

In general, the present disclosure relates to devices and methods for temporary vascular occlusion for controlling major and/or traumatic internal bleeding in patients. The systems described herein include a delivery device and a guidance system.

### Delivery Device and System

In a first embodiment, the devices described herein include a delivery or catheter device with a biodegradable temporary occlusion agent. In another embodiment, the device is a delivery system that includes a delivery or catheter device with a biodegradable temporary occlusion agent, where the delivery system, such as a catheter, is further utilized as a vascular access device (or intravascular catheter device) for monitoring blood pressure (or other biosensor, such as heart rate, blood oxygen saturation levels, or other similar vital sign). Still further, the catheter device may be used to deliver other agents prior to or subsequent to the delivery of the biodegradable temporary occlusion agent or removable occlusion product. Other agents may include fluids, blood, or other therapeutic fluids for the patient.

The delivery device or catheter described herein is designed to be used without fluoroscopy. As described in more detail below, the delivery device(s) include a communication or location device that can communicate with an external receiver. The external receiver helps guide the insertion of delivery device to the appropriate treatment location within the vessel for deployment of a temporary embolic agent.

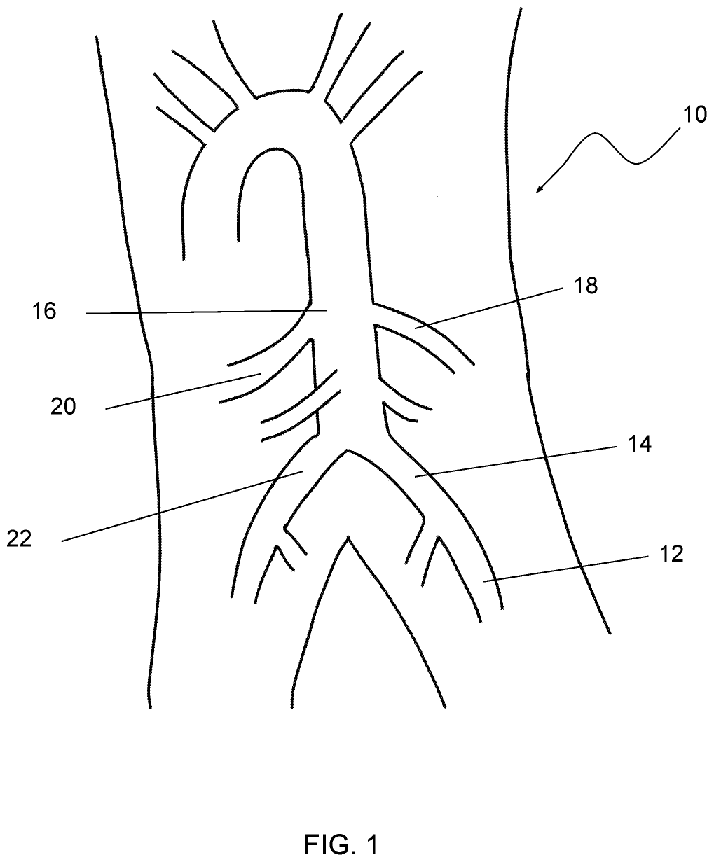

FIG. 1 illustrates an example anatomical representation of the major blood vessels in the human abdomen. A simplified normal arterial tree 10 with major branch artery points is illustrated. The major branch artery points include a left femoral artery 12 and an external iliac artery 14. Other major branch artery points include the celiac artery origin 16, the left renal artery origin 18, the right renal artery origin 20, and the aortic bifurcation 22. It should be noted that many other vessels exist in the normal arterial tree 10 that are not shown.

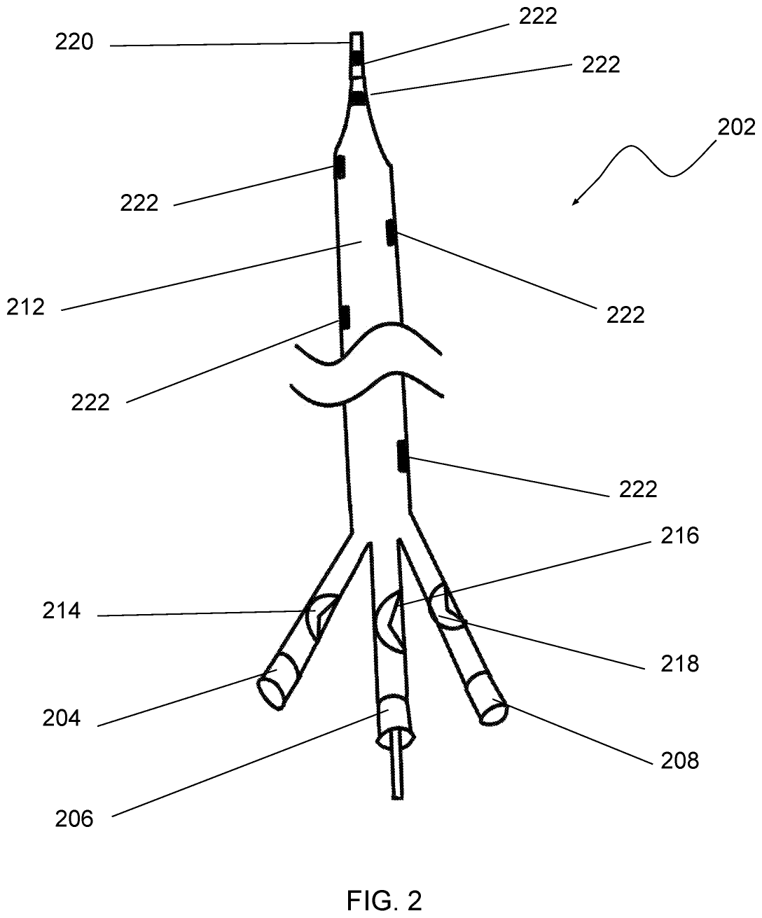

FIG. 2 illustrates an example embodiment of a catheter device 202 for supplying a temporary occlusion agent. The delivery or catheter device 202 may be used for positioning, delivering, and/or deploying a vessel occlusion device and/or a vessel occlusion agent. As described in more detail below, a vessel occlusion device may be a mechanical occlusion device while a vessel occlusion agent may be a chemical composition. One of skill will understand that the catheter device 202 described herein may include fewer elements, for example, when the catheter device 202 is only used for positioning, or for example, when the catheter device 202 is only used for positioning and deploying a vessel occlusion agent, but not delivering a vessel occlusion device.

In an example embodiment, the catheter device 202 includes a shaft 212, three access ports 204, 206, 208, and a tip portion 210. Other catheter devices contemplated may include more or less than three access ports. In a first embodiment, each access port 204, 206, 208 is in fluid communication with an individual lumen within the catheter device 202 body. In an alternative embodiment, each access port 204, 206, 208 is in fluid communication with a single lumen of the catheter device 202.

The first access port 204 can be used for vascular access, for example, to transfer fluids such as blood, medication, or saline into a vessel. The second access port 206 can be used for threading over a guidewire 220. The third access port 208 can be used to supply the temporary embolic agent. Each access port 204, 206, 208 also includes a stopper 214, 216, 218, capable of opening the lumen existing in each access port 204, 206, 208.

As stated, the first access port 204 can be used to transfer fluids into a vessel. Example types of liquid fluids may be blood, such as for a blood transfusion; medication, such as epinephrine, vasopressin, dopamine, volume expanders, saline, lactated Ringer's solution, fresh frozen plasma, packed red blood cells, platelets, or other blood products. This list is not to be seen as determinative.

The second access port 206 is configured for being inserted over a guidewire 220. The guidewire 220 can be a traditional or standard guidewire normally used to insert a catheter within a blood vessel. For example, guidewire 220 may be a solid core wire, or a core wire wrapped with numerous smaller braided wires or wire coils surrounding the core wire. The core wire may be hollow. Still further, the guidewire 220 may be coated with a polymer coating such as silicone or polytetrafluoroethylene (PTFE).

Additionally, the guidewire 220 includes communicative functionality. Such communicative features allow a healthcare provider to insert the guidewire 220 without the use of fluoroscopy or x-ray. The guidewire 220 includes a communication device 222 or biosensor at a distal end, wherein the communicate device 222 can communicate with a receiver external to the patient. A communication device 222 as described herein is referred to generally as a technological tag that allows a healthcare provider to insert a guidewire 220 within a patient and know the location of the guidewire 220 without relying on fluoroscopy or x-ray technology.

The communicative or location device 222 may be attached to the distal end of the guidewire 220 with any known technique, such as but not limited to, adhesives, fasteners, integral molding, or other technique.

Communicative or location devices 222 are located at least at the distal end of the guidewire 220, but may be located along the length of the guidewire 220 at distinctive intervals. In an example embodiment, the communicative or location devices 222 are located at the distal end of the guidewire 220 and at equally spaced intervals along the length of the entire guidewire 220.

The communication or location device 222 may be an echogenic marker that is capable of communicating with a doppler receiver, where an external doppler receiver can identify the location of the guidewire 220 (or the tip portion 210 if a guidewire 220 is not used, as described in more detail below) by sensing the location of the communication device 222. In another embodiment, a communication device 222 may include RFID or NFC technology, which is capable of communicating with a computing device or an insertion template 300 (described in more detail below) or other receiver that helps the healthcare provider place the catheter device 202 in the correct location. In still yet another embodiment, the communication device 222 may include infrared technology or temperature sensor technology.

The communication or location device 222 may communicate with an external receiver in a variety of ways. For example, the communication device 222 may wirelessly transmit a signal to a smart phone or other computing device with or without a screen. In such an example, similar to reading a fluoroscopy screen, as the healthcare provider is inserting the guidewire 220 into the blood vessel of the patient, the location of the guidewire 220 is displayed on the computing device, so the healthcare provider knows where the guidewire 220 is located.

In another example, the communication device 222 may include a built-in notification device such as an auditory signal that can work in conjunction with a smart phone or other computer device. When the guidewire 220 is nearing a final location within the blood vessel, a signal is produced notifying the healthcare provider of the final location of the guidewire 220.

Still further, and described in more detail below, the communication device 222 may work with a robotic device and/or artificial intelligence (AI) enhanced technology. The communication devices 222 may communicate with an external receiver that helps guide the delivery device 200 to a desired location. This process could aid a healthcare provider, or alternatively, be used to guide the delivery device 200 without the active deployment from a healthcare provider.

An automated delivery device 200 may include additional elements that allow for the automation of deployment of the temporary embolic agent. In an example, the delivery device 200 may include a reel, motor, and/or battery that allows the device to automatically deploy a temporary embolic agent without a healthcare provider actively deploying it. For example, after the delivery device 200 is located at a final position within a blood vessel, the healthcare provider can push a button (not shown) that activates the motor to deploy the temporary embolic agent.

Alternatively, or in addition to a guidewire 220, the catheter 202 may include a tip portion 210 that includes a communication device 222. In a first embodiment, a communication device 222 may include doppler technology, where an external doppler receiver can identify the location of the tip portion 210 by sensing the location of the communication device 222. In another embodiment, a communication device 222 may include RFID or NFC technology, which is capable of communicating with an insertion template 300 or other receiver that helps the healthcare provider place the catheter device 202 in the correct location. However, other similar communication devices and technologies may be utilized.

In addition to or alternatively, one or more communication devices 222 may be located along the shaft 212. The communication device(s) 222 may be staggered along the shaft 212, symmetrical along the shaft 212, or located in other patterns. As stated above, the communication device 222 may include RFID or NFC technology, which is capable of communication with an insertion template 300 or other receiver that communicates with the selected technology. However, other similar communication devices and technologies may be utilized.

The third access port 208 is useful for supplying the temporary embolic agent. The temporary embolic agent may be selected from any biocompatible injectable agent that occludes a blood vessel and degrades after a period of time. The selected temporary embolic agent degrades, which allows restoration of blood flow through the blood vessel without the removal of an otherwise permanent implant and therefore, no further injury or further procedure to the patient is required.

In still a further embodiment, the catheter device 202 may have a handle (not shown) located at a proximal end. The handle can include an actuation mechanism that facilitates the deployment of the temporary embolic agent. The handle may or may not include a sensor display. A sensor display may communicate with communication devices 222 that enable a healthcare provider to accurately guide the catheter device 202 to a target location within a vessel.

In a first embodiment, the temporary embolic agent may be delivered as a liquid or gel via a syringe through the catheter device 202. In a second embodiment, the temporary embolic agent may be delivered as a gel or foam via a canister delivered with the catheter device 202. In another embodiment, and as described in more detail below, the temporary embolic agent may be delivered within a capsule or other similar device. In yet another embodiment, the temporary embolic agent may be a particulate embolic, such as a microsphere with or without a medium.

Temporary embolic agents are selected based on biocompatibility and time to degrade. Such temporary embolic agents may be selected from degradable sugar-based molecules such as sorbitol, polysorbate, polyethylene glycol, and acetyltributyl citrate. Other type of temporary embolic agents may be selected from the list including, but not limited to, degradable starch microspheres, poly(lactic-co-glycolic acid), PLGA-Polyethylene Glycol-PLGA, carboxymethylcellulose, chitin, hydroxyethyl acrylate, albumin, gelatin, pluronic F127, polyvinyl alcohol, cellulose acetate (CA), polylactic acid (PLA), poly(glycolic acid) (PGA), copolymers of the PLA and PGA, polycaprolactone (PCL), polycaprolactone (PCL), poly(L-lactide-co-ε-caprolactone) (PLCL), and poly(D,L-lactide-co-glycolide) (PLGA). Other types of semi-crystalline and amorphous homopolymers may also be utilized. Other types of temporary embolic agents may also be utilized.

The temporary embolic agent is selected to occlude the blood vessel(s) for a predetermined time period before dissolving on its own. Then, the healthcare provider does not need to actively remove the embolic agent. The temporary embolic agent may occlude a blood vessel for about five minutes to about six hours. In an alternative embodiment, the temporary occlusion agent may occlude a blood vessel for about five minutes to about 30 minutes, for about 30 minutes to two hours, about two hours to about four hours, or from about three hours to about six hours. However, these time periods are not to be seen as limiting.

Although not shown, the catheter device 202 may include additional monitoring sensors. For example, the catheter device 202 may include a pressure monitor, art line function, CO2 monitor, pH monitor, or other similar biosensors to monitor vital signs of the patient. The biosensors may aid in the insertion of catheter device 202, such as sensors useful for bio-navigation, positioning, or communication with robotic devices/AI enhanced technology. Such sensors may include ultrasound, thermal, electromagnetic, or other similar sensors.

The biosensors may be located in similar locations to the communications devices 222, where the sensors are located along the shaft 212. For example, one or more sensors may be located along the shaft 212. The sensors may be staggered along the shaft 212, symmetrical along the shaft 212, or located in other patterns.

In an exemplary embodiment, the catheter device 202 is sized and shaped to be inserted within the femoral artery and extend towards the distal abdominal aorta. The catheter device 202 may have a shaft 212 with a length of at least 15 cm, for example about 35 cm; however other lengths are contemplated. An outer diameter of the catheter device 202 may be from about 30 mm to about 75 mm; however, other outer diameters are contemplated.

The catheter device 202 may be made from standard or traditional materials, such as a flexible biocompatible plastic material. Other materials are also contemplated, such as materials that allow for internal steerability or materials that are shapeable or have shape-memory properties.

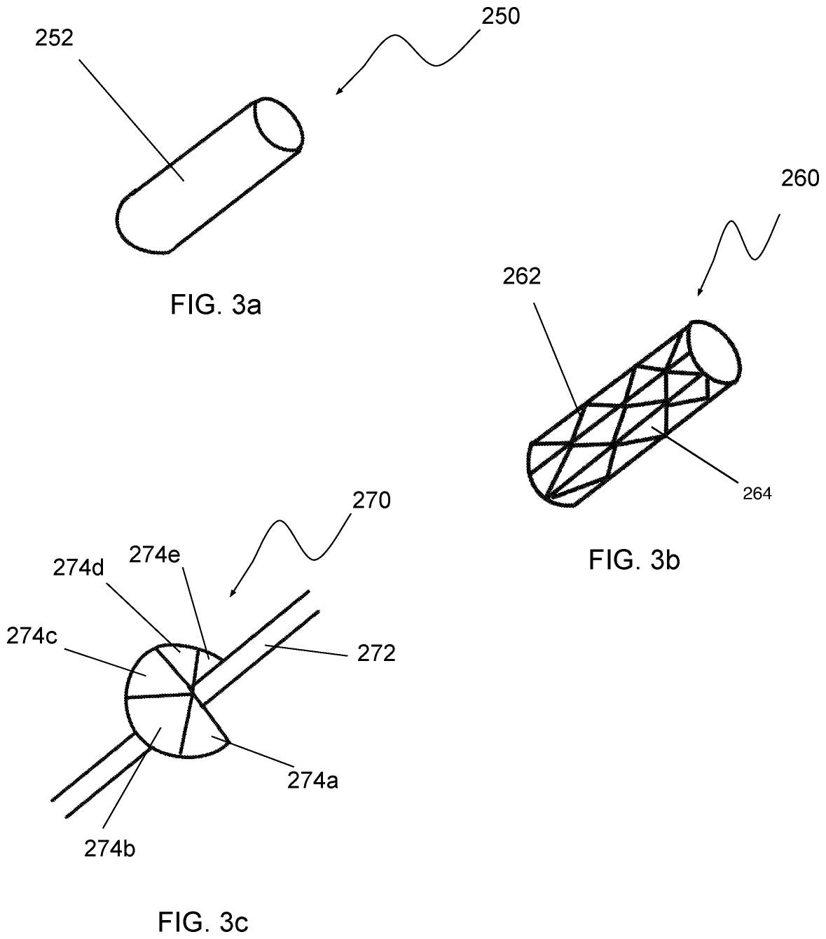

FIG. 3a illustrates an example embodiment of an embolic agent device 250. The embolic agent device 250 includes an external occlusive wafer 252 that houses the temporary embolic agent. The embolic agent device 250 can be delivered via the catheter device described above. When the occlusive wafer 252 is moistened or otherwise subjected to liquid, the occlusive wafer 252 dissolves so the temporary embolic agent can occlude the blood vessel. The composition of the temporary embolic agent is discussed above.

The occlusive wafer 252 can be made from various biocompatible materials that rapidly disintegrate. An example material is water soluble polymers, while other materials may be used. Other materials may include: starch, modified starch, poly(lactic-co-glycolic acid) PGLA, PLGA-polyethylene, glycol-PLGA, carboxymethylcellulose chitin, cellulose, hydroxyethyl acrylate albumin, gelatin, pluronic F127, polyvinyl alcohol, hyaluronic acid hydrogel, poly(ethylene glycol) methacrylate (PEGMA), bioresobable silicone, chitosan/konjac glucomannan matrix and other starch-based molecules.

While the embolic agent device 250 is shown in as pill-shape, other shapes are contemplated. The embolic agent device 250 may be spherical or disc shaped. The embolic agent device 250 may be sized from about 10 micron to about 1000 micron in diameter.

In use, a healthcare provider may be able to deliver as many embolic agent devices 250 as needed. In a first example, a catheter device 202 may be preloaded with multiple numerous embolic agents 250 and is able to deploy them as needed. In an alternative example, the healthcare provider may be able to reload the catheter device 202 as many times as needed.

FIG. 3b illustrates another example of an embolic agent device 260. The embolic agent device 260 may include an external self-expanding scaffold stent 262 that houses the temporary embolic agent within a membrane 264. The external self-expanding scaffold stent 262 and/or membrane 264 is biocompatible and may be bioabsorbable. The self-expanding scaffold stent and/or membrane 264 has a matrix that allows for blood flow after the temporary embolic agent has finished occluding a blood vessel. Then, it is not necessary that it be removed later. However, the scaffold stent 262 may be removed at a later time.

FIG. 3c illustrates yet another example of an embolic agent device 270. Embolic agent device 270 is useful for incomplete vessel occlusion and may be used with or without a temporary embolic agent. Embolic agent device 270 includes an elongated central portion 272 with a plurality of plates 274a-e that extend perpendicular therefrom. The plurality of plates 274a-e may include the temporary embolic agent. For example, the temporary embolic agent may be adhered to a surface of the plurality of plates 274a-e. The embolic agent device 270 may include at least one set of the plurality of plates 274a-3, for example, including two sets of a plurality of plates 274a-e that extend at different levels along the elongated central portion 272.

The plurality of plates 274 may be a solid surface, or alternatively, may have a porous surface. Still further, the plurality of plates 274 may not be bioabsorbable, but form an incomplete occlusion after complete occlusion need is met, allowing for the return of blood flow through the vessel. Alternatively, the plurality of plates 274 may be made from a membrane that is bioabsorbable, such as being made from various biocompatible materials that rapidly disintegrate. An example material is water soluble polymers, while other materials may be used.

The plurality of plates 274 may extend completely circumferentially around the elongated central portion 272 for complete occlusion of a blood vessel. Alternatively, the plurality of plates 274 may extend incompletely circumferentially around the elongated central portion 272 for partial occlusion of a blood vessel.

In a further example embodiment, the elongate central portion 272 includes pressure sensors 276. The pressure sensors 276 provide hemodynamic monitoring feedback to the user and the control unit which can autonomously or semi-autonomously regulate the amount of blood flow blockage by opening or closing the plates to optimize patient hemodynamics.

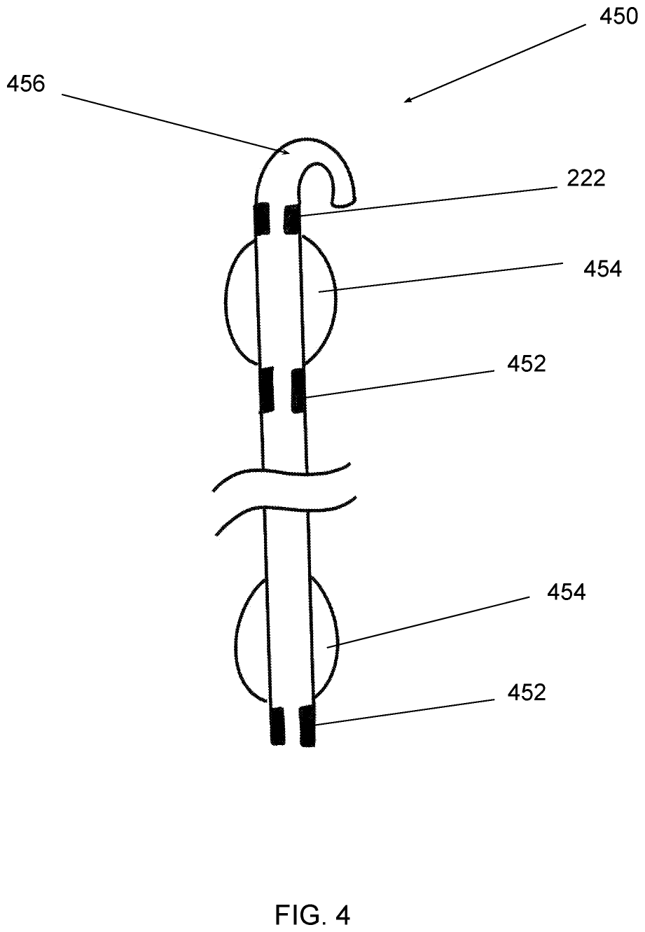

FIG. 4 illustrates yet another delivery device 450 for temporary embolic agent delivery to a target blood vessel. The delivery device 450 includes a plurality of pressure sensors 452 located along a length of the device. The pressure sensors 452 may function the same or similar to the pressure sensors described above.

The delivery device 450 also includes at least one automated balloon 454, configured to example the entire circumference of the blood vessel to hold the delivery device 450 in place while the temporary embolic agent is deployed within the blood vessel. The balloons 454 can be deflated once the temporary embolic agent is deployed, and the entire delivery device 450 can be removed or left in place for additional treatment, ongoing hemodynamic monitoring, or other similar needs.

Balloon 454 also provides occlusion of the aorta by stopping blood flow to bleeding site and/or allowing for infusion of embolic above, below, or in between the occlusion balloons. The balloon(s) 454 prevent the temporary embolic agent from reaching the non-target arteries excluded by the balloons. In an example, a proximal balloon 454 could be inflated at the level of the SMA and a distal lower balloon 454 just below the renal arteries. If the temporary embolic agent is infused through the port between the balloons all the temporary embolic agent is directed to the renal arteries.

In a further embodiment, internal diagnostics on the catheter can diagnose where the bleeding is coming from; therefore, allowing for selective temporary embolic agent deployment without angiography.

The distal end 456 can include one or more communication devices 222 (although it should be noted that multiple communication devices 222 may be located along the length of the catheter device). As described above, the communication device 222 may include RFID or NFC technology, which is capable of communication with an insertion template 300 or other receiver that communicates with the selected technology. However, other similar communication devices and technologies may be utilized.

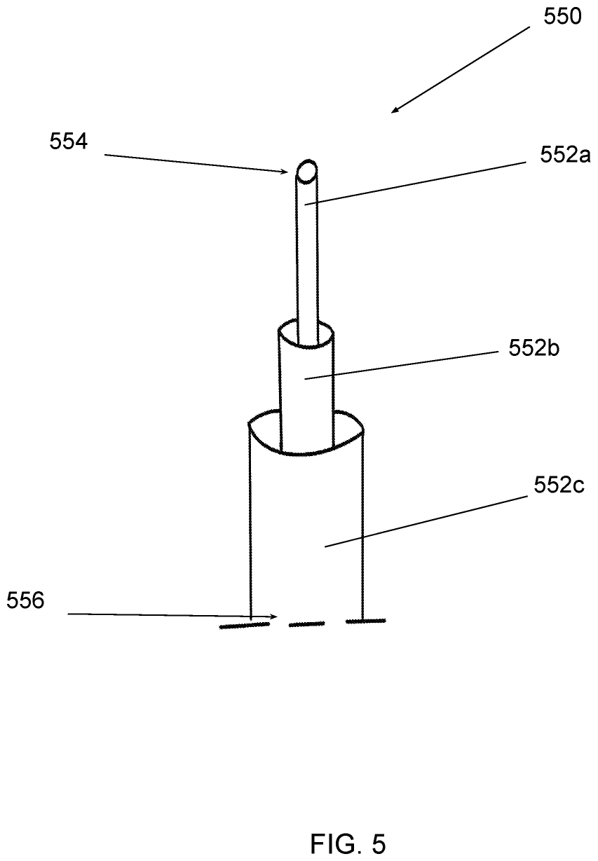

FIG. 5 illustrates yet another example delivery device 550. The delivery device 550 includes a plurality of concentric telescoping sheaths 552a, 552b, 552c. A distal end 554 of the inner most telescoping sheath 552a includes the temporary embolic agent (not shown). A handle (not shown) located at a proximal end 556 includes a push insertion button that a healthcare provider can use to insert the distal end 554 to a desired location within a blood vessel.

The plurality of concentric telescoping sheaths 552a, 552b, 552c and/or distal end 554 may further include sensors to help guide the delivery device 550 up an artery and may be deflectable (like deflecting guidewire) to help steer. It should be noted that each concentric telescoping sheath 552 may include a smooth taper between components.

The distal end 554 can include one or more communication devices 222. As described above, the communication device 222 may include RFID or NFC technology, which is capable of communication with an insertion template 300 or other receiver that communicates with the selected technology. However, other similar communication devices and technologies may be utilized.

In an embodiment, one of the plurality of telescoping sheaths 552a, 552a, 552c may include a balloon (not shown) configured to example the entire circumference of the blood vessel to hold the delivery device 550 in place while the temporary embolic agent is deployed within the blood vessel. The balloon(s) can be deflated once the temporary embolic agent is deployed, and the entire delivery device 550 can be removed or left in place for additional treatment, ongoing hemodynamic monitoring, or other similar needs.

The delivery device 550 may also be used with a guidewire 220 as described above.

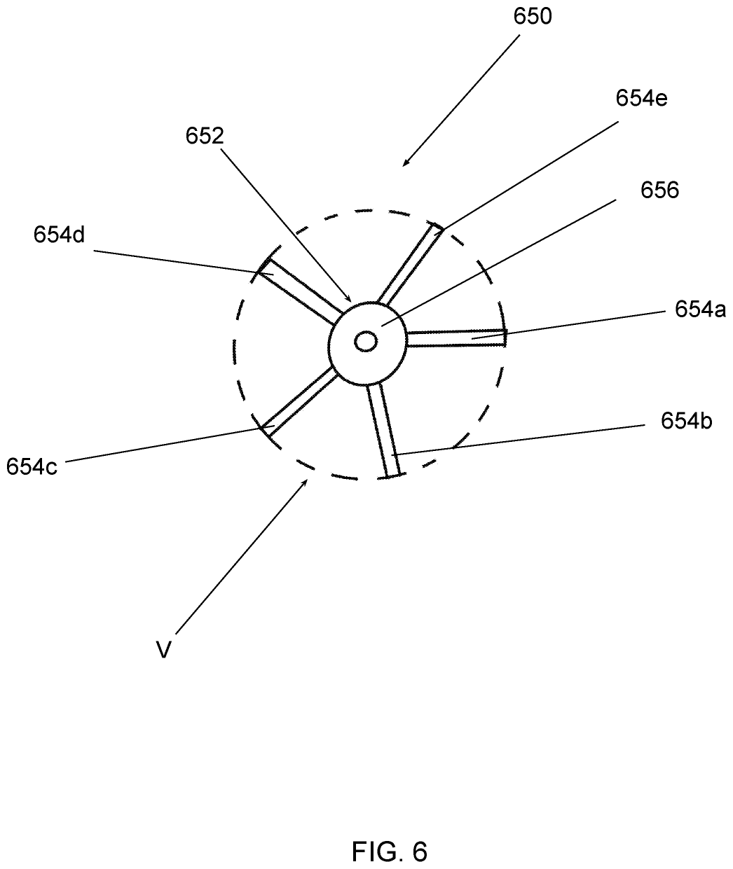

FIG. 6 illustrates a top-down view of another example delivery device 650 with an expanding distal end 652. The delivery device 650 includes a central catheter 656 through which the temporary embolic agent may be deployed. The expanding distal end 652 may be a collapsible and/or flexible “umbrella” that, when extended, occludes a blood vessel. The distal end 652 may have a plurality of extending tines 654a-e that make up the “umbrella” shape. When the tines 654a-e extend within the blood vessel V, the central catheter 656 is centered within the blood vessel V. Centering within a blood vessel V is advantageous when guiding the delivery device 550 without angiographic guidance.

The delivery device 650 may also include a balloon (not shown) configured to example the entire circumference of the blood vessel to hold the delivery device 650 in place while the temporary embolic agent is deployed within the blood vessel. The balloon(s) can be deflated once the temporary embolic agent is deployed, and the entire delivery device 550 can be removed.

It should be noted that the expanding distal end 652 may be located on the catheter 202 described above. The delivery device 650 may also be used with a guidewire 220 as described above.

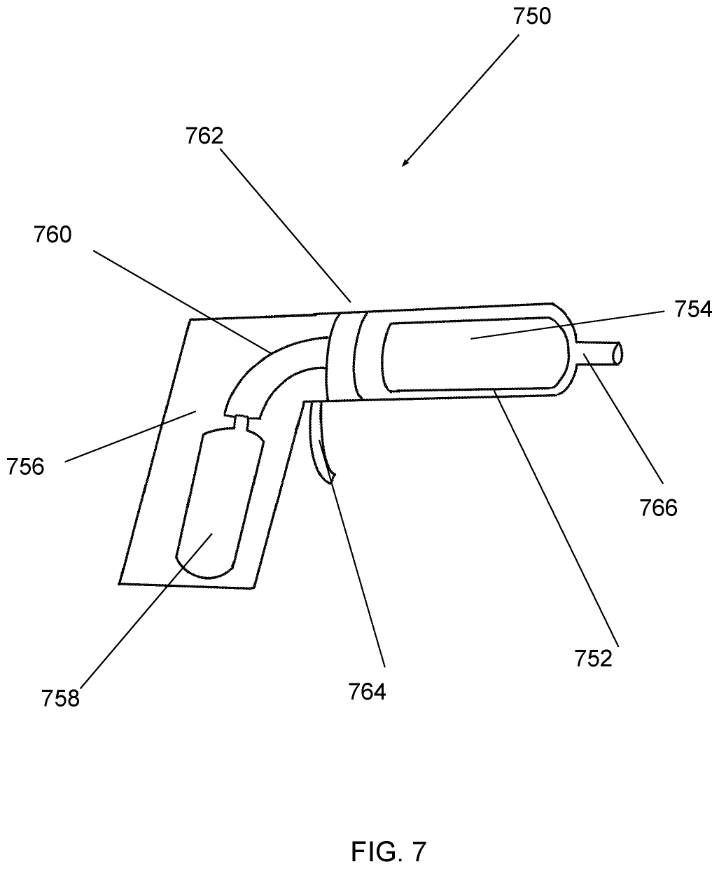

FIG. 7 illustrates an example trigger style delivery device handle 750. Delivery device handle 750 can be connected to a cather, such as the one described above at access portion 766.

Delivery device 750 includes a barrel 752 containing the temporary embolic agent 754 and a handle portion 756. Handle portion 756 includes a CO2 cartridge 758, which acts as the deployment mechanism. Upon actuation of the trigger 764 the CO2 travels from the cartridge 758 through the conduit 760 to depress the plunger 762. The plunger 762 pushes the temporary embolic agent 754 through the access port 766.

### Guidance System

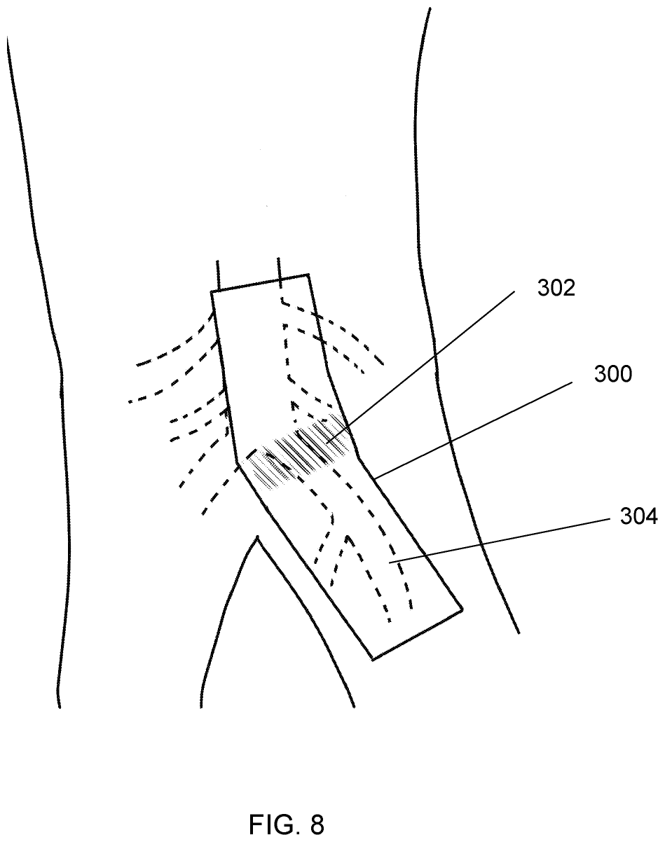

FIG. 8 illustrates an example embodiment of an insertion template 300. As described, the catheter device 202 or delivery device 550, 650 is inserted within a patient with the use of a communication device 222. The communication 222 is configured to be sensed by an external receiver. One such external receiver may be an insertion template 300 that is placed on a patient.

The insertion template 300 includes anatomical landmarks useful for navigating the guidewire 220 and associated catheter 202 device when fluoroscopy or x-ray is unavailable. The insertion template 300 also includes indicia indicating the desired delivery location 302 of the temporary embolic agent.

The insertion template 300 can have an adhesive on a first side, which is capable of releasably sticking to the skin of the patient. In an example, a healthcare provider can place the insertion template 300 right below the sternum of the patient. Such a location can approximate the delivery location 302 of the temporary embolic agent. In another example, the insertion template 300 can be placed at the femoral head or other bony landmark to approximate the delivery location 302 of the temporary embolic agent. The insertion template 300 may also include various other sensors to assist with bio-navigation.

Although not shown, the insertion template 300 may also include other visual indicators or displays that help to provide feedback to the healthcare provider. Further, the insertion template 300 may include sensors or other communication device 222 useful to identify anatomical locations, such as the location of an artery. This indication can be used by a healthcare provider and/or AI systems such as robotics to steer the delivery device to the correct position within an artery.

In an example insertion template 300, the desired delivery location 302 of the temporary embolic agent includes a sensor that can communicate with the communication device 222 of the guidewire 220 or the tip portion 210. When the guidewire 220 and/or tip portion 210 is nearing the delivery location 302, the healthcare worker can be alerted via a sound, light, or other similar means.

In an exemplary example, the communication protocol is NFC technology. An alert such as sound or light, may be activated when the sensor of the insertion template 300 detects the NFC tag of the guidewire 222. Other similar technologies may also be used.

While shown on the left side of a patient, an insertion template 300 could also be used for a right side of a patient. Still further, the insertion template 300 could be designed to cover both the left and right side of the patient.

Further, while the insertion template 300 is shown as a template that is only sized to indicate a location of the femoral artery, the insertion template 300 may be larger and may lay over a patient, such as a blanket would, without the use of an adhesive. The healthcare provider can lay the blanket-type device over the patient using anatomical markers to place it at the correct location, such as laying an edge of the blanket-type device directly below the sternum of the patient.

Still further, the insertion template 300 may be a sleeve that can be placed around the patient, such as around a leg or a torso. A sleeve-like insertion template 300 may or may not include an adhesive. In an example, an edge of the sleeve can be placed on a limb as close to the torso as possible. The sleeve can approximate the anatomical landmarks of a patient, to approximate the delivery location 302 of the temporary embolic agent. A sleeve may also include various other sensors to assist with bio-navigation.

In an example embodiment, a sensor system (not illustrated) can communicate with robotics/AI enhanced technology to use recent CT or MRI imaging from the patient (or similar-sized patient) as the navigation template with or without the use of the insertion template 300. The position of the catheter device 202 can be calculated by AI using the CT and/or MRI anatomical data and anatomical measurements. The position of the guidewire 220 and/or tip portion 210 could be displayed in multiple ways such as augmented reality, conventional monitor screen, smart phone, or similar computing device. Such a sensor system allows AI/robotics and/or the healthcare provider to move the catheter device 202 through blood vessels with minimal or no need for X-Ray or fluoroscopy.

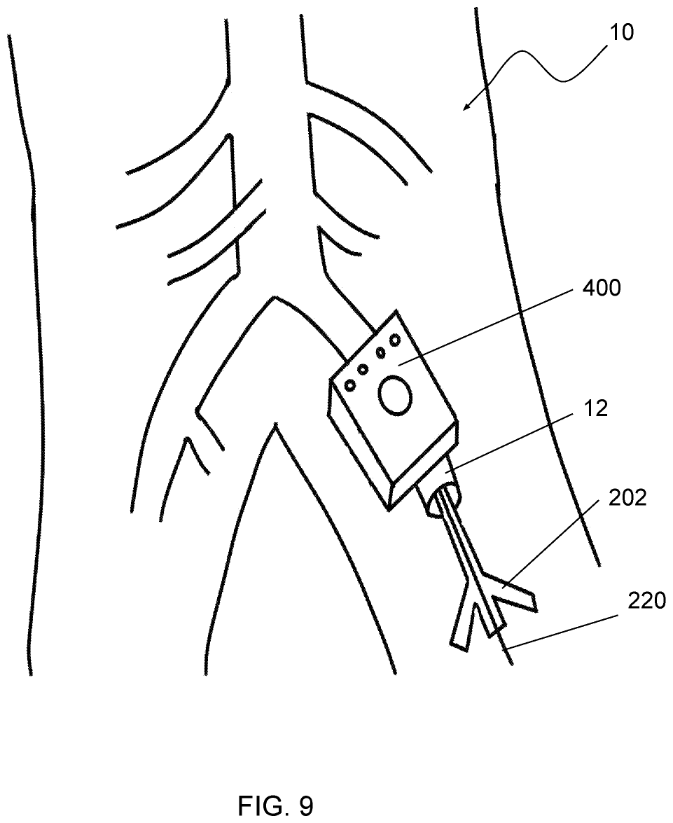

FIG. 9 illustrates an example embodiment of insertion of the catheter device 202 or delivery device 550, 650 with a doppler 400. A doppler 400 may be used instead of an insertion template 300. A healthcare provider inserts the guidewire 220 with the help of doppler 400. The guidewire 220 includes echogenic markers as the communication device 222. The doppler 400 is capable of sensing the communication device 222 of the guidewire 220. Then, the healthcare provider can insert the catheter device 202 over the guidewire 220 to the desired delivery location within the vessel of the patient.

Alternatively, or in addition to the guidewire 220, the tip portion 210 of the catheter device 202 may be identifiable by doppler 400, so the catheter device 202 is capable of being placed without a guidewire 220.

In an alternative embodiment, a NFC receiver may be used as the doppler is described above. As shown in FIG. 4, instead of the doppler, a NFC receiver (not shown) communicates with the communication device(s) 222 of the guide 220 for a desired placement within a blood vessel.

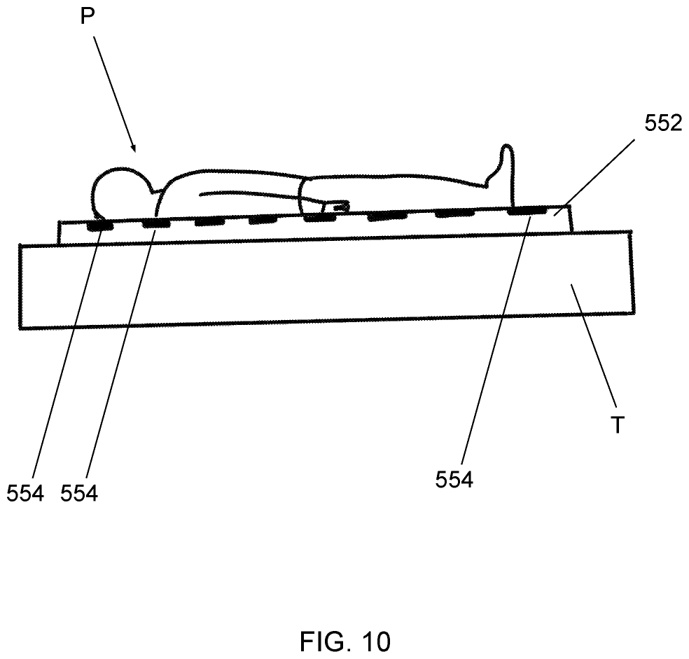

FIG. 10 illustrates another device for aiding in insertion of the catheter device 202 when traditional methods, such as fluoroscopy or x-ray are not available. FIG. 9 shows a patient P on a table T, such as an operating room table, or other similar table. Included on table T is a sensor pad 552. The sensor pad 552 includes a plurality of communication devices 554 that are capable of communicating with communication devices 222 of the guidewire 212. The communication devices 554 may include NFC technology, which is capable of communicating with a guidewire 212 or a tip portion 210, or other sensor that helps the healthcare provider place the catheter device 202 in the correct location.

In still yet another embodiment, the communication device 554 may include infrared technology or temperature sensor technology. However, other similar communication devices may be utilized.

In yet another embodiment, the sensor pad 552 may communicate with the insertion template 300 to aid in insertion of the catheter device 202.

### Methods of Use

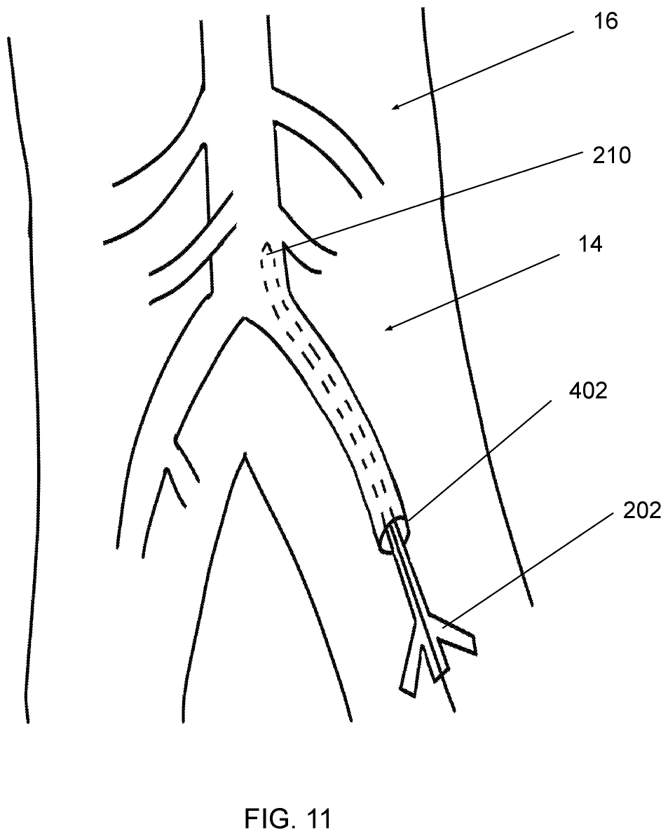

FIG. 11 illustrates the catheter device 202 within a blood vessel V. Delivery device 450, 550 may also be utilized in the methods described herein. As shown, the catheter device 202 is inserted within an incision 402 or accessible location of the blood vessel of the patient. As described below in more detail, the catheter device 202 is inserted within the blood vessel, until the tip portion 210 is at a desired delivery location.

Although not shown, the catheter device 202 and/or guidewire 222 may include indicia that indicate the length of the catheter. Indicia may be numerical, where the numbers start at a distal end of the catheter and extend proximally. A healthcare provider can use the indicia as a guide for the length of the catheter device 202 that is inserted in the blood vessel.

The indicia may be, additionally or alternatively to numbers, color coded, where a specified color indicates that the tip portion 210 and/or guidewire 222 of the catheter device 202 is at a specific anatomical location within the blood vessel. For example, a first color may indicate the tip portion 210 and/or guidewire 222 is inserted at a length generally extending to the aortic bifurcation. A second color may indicate the tip portion 210 and/or guidewire 222 is inserted at a length generally extending to the renal arteries. A third color may indicate the tip portion 210 and/or guidewire 222 is inserted at a length generally extending to the supra celiac artery or the diaphragm. However, the catheter device 202 and/or guidewire 222 may include more or less colors.

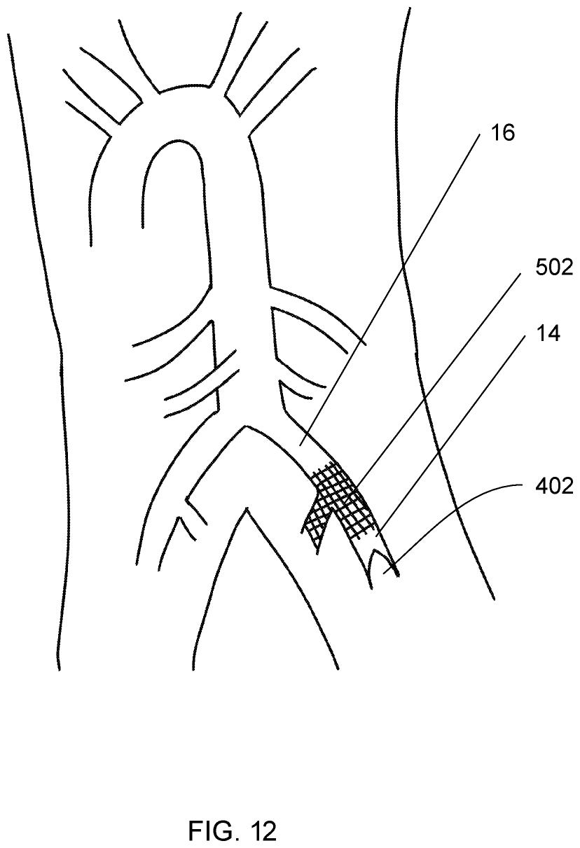

FIG. 12 illustrates an example of the temporary embolic agent 502 dispersed with the blood vessel V. As shown, the temporary embolic agent 502 extends within the entire blood vessel to completely occlude the blood vessel and stop any potential blood flow.

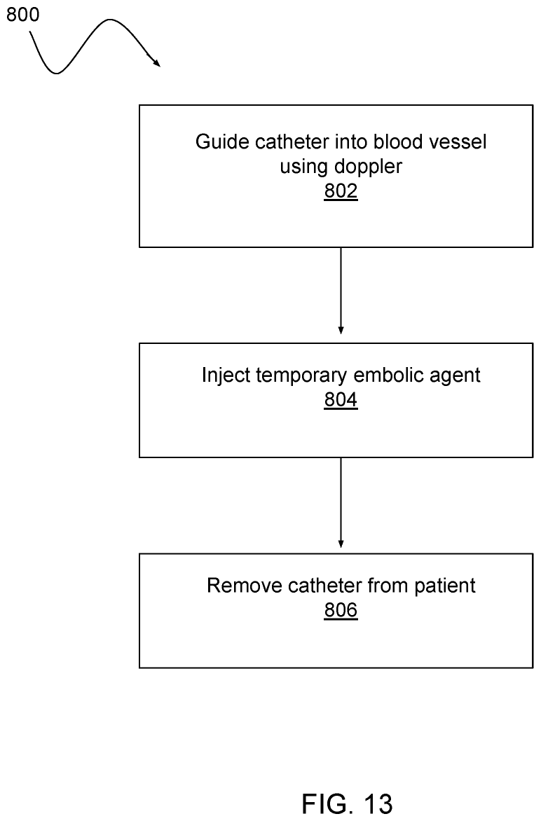

FIG. 13 illustrates and example method 800 of use of the catheter device 202. In the example method, a doppler is selected as the communication technology; however, any communication protocol may be used as described by method 800.

At step 802, the healthcare provider uses a doppler to guide the catheter device 202 into the blood vessel. The communication sensor 222 is a doppler sensor on the guidewire 220 or tip portion 210 that is detectable by doppler to ensure the guidewire 220 is guided properly into the blood vessel. After the guidewire 220 is positioned at a desired location, the catheter 202 is inserted over guidewire 220. Guidewire 220 can then be removed.

At step 804, the temporary embolic agent 502 is injected into the patient via the catheter device 202. The different types of temporary embolic agents are described above, and are selected based on their biocompatibility and time to degrade within the human body.

At step 806, the catheter device 202 is removed from the patient, while the temporary embolic agent 502 remains within the blood vessel and completely occludes the blood vessel.

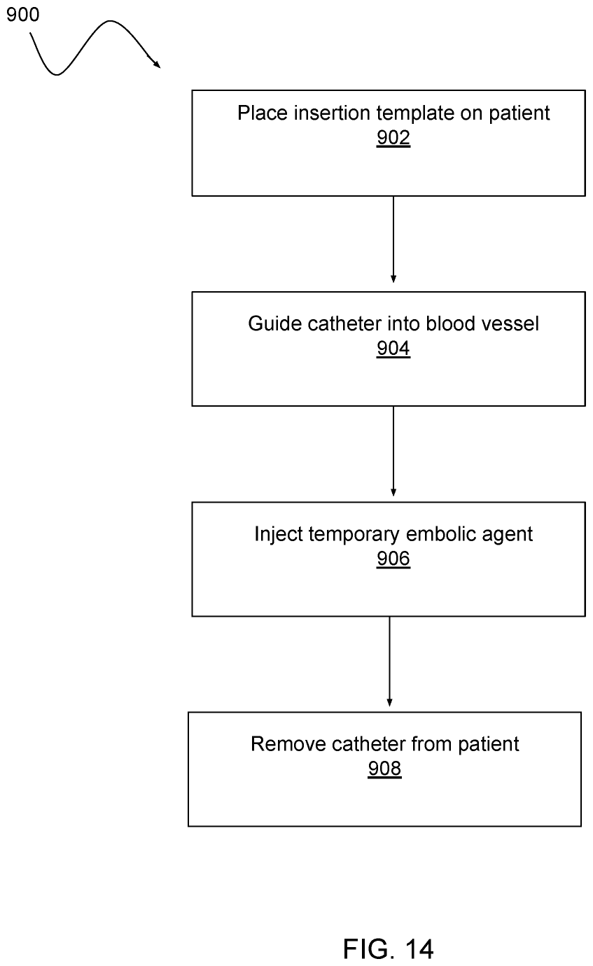

FIG. 14 illustrates and example method 800 of use of the catheter device 202 and insertion template 300. Delivery device 450, 550 may also be utilized in the methods described herein. In the example method, an insertion template 300 is selected as the communication technology; however, other guidance system may be used as described by method 900.

At step 902, an insertion template 300 is placed on the skin of a patient. The insertion template 300 can be placed as directed, such as being placed at the base of the patient's sternum and then extending all the way to the mid-thigh. The insertion template 300 includes a communication receiver that provides guidance to the healthcare provider for insertion of the guidewire 220 and/or catheter device 202 through a blood vessel. In a non-limiting example, the blood vessel is the femoral artery extending to the descending aorta. However, other blood vessels may be targeted for delivery of a temporary embolic agent.

At step 904, the guidewire 220 is navigated within the blood vessel to a desired final location via communication between the communication device(s) 222 and insertion template 300. After the guidewire 220 is positioned at a desired location, the catheter 202 is inserted over guidewire 220. Guidewire 220 can then be removed.

Alternatively, tip portion 212 may include the communication device(s) 222, so a guidewire 220 is not required. The tip portion 212 is navigated within the blood vessel to a desired final location via communication between the communication device(s) 222 and insertion template 300.

At step 906, the temporary embolic agent 502 is injected into the patient via the catheter device 202. The different types of temporary embolic agents are described above and are selected based on their biocompatibility and time to degrade within the human body.

At step 908, the catheter device 202 is removed, while the temporary embolic agent 502 remains within the blood vessel and completely occludes the blood vessel for a limited time period. After the catheter device 202 has been removed, the insertion template 300 can also be removed.

In another example method, the method of FIG. 9 is performed with a computer assisted robotic device. The insertion template 300 may include sensors capable of communicating with the computer assisted robotic device to accurately guide the catheter device 202 into the blood vessel to a desired delivery location.

It should be noted that the catheter device 202 may also be inserted via traditional methods, such as placement with fluoroscopy or x-ray.

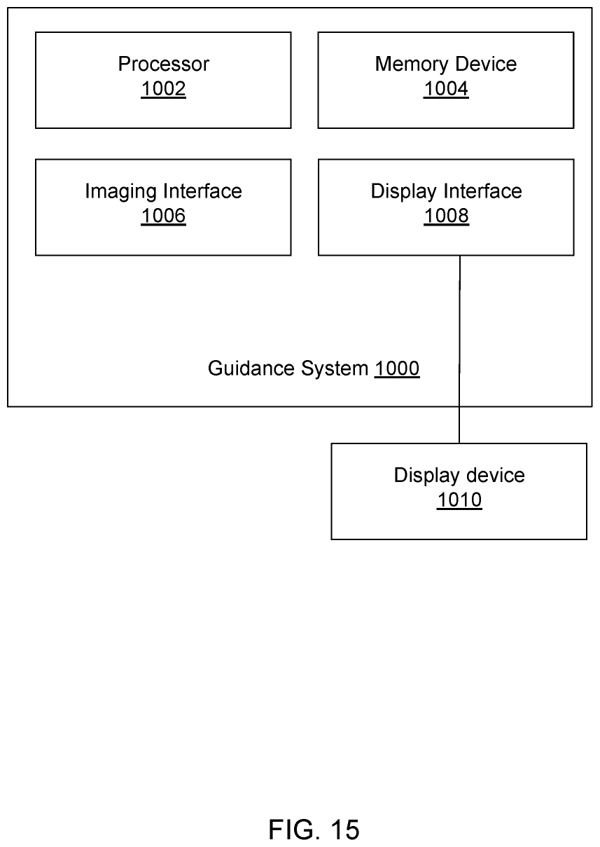

FIG. 15 illustrates and an example computing system used as part of the guidance system. In an example, the guidance system 1000 comprises an imaging interface that can receive imaging data from a plurality of sources (as described above). A guidance system 1000, generally, includes one or more processors 1002, one or more memory devices 1004, an imaging interface 1006, and a display interface 1008.

The one or more processors 2002 includes any conventional processor, such as a commercially available CPU. In other embodiments, the processing device additionally or alternatively includes one or more digital signal processors, field-programmable gate arrays, or other electronic circuits.

The memory device 1004 typically includes at least some form of computer-readable media. By way of example, computer-readable media include computer readable storage media and computer readable communication media. The memory device 1004 includes instructions that, when executed by the processor 1002 cause the one or more processor to perform operations that aid in positioning the catheter device 202.

Computer readable storage media includes volatile and nonvolatile, removable and non-removable media implemented in any device configured to store information such as computer readable instructions, data structures, program modules, or other data. Computer readable storage media includes, but is not limited to, random access memory, read only memory, electrically erasable programmable read only memory, flash memory and other memory technology, compact disc read only memory, blue ray discs, digital versatile discs or other optical storage, magnetic storage devices, or any other medium that can be used to store the desired information and that can be readily accessed. In some embodiments, computer read-able storage media is non-transitory computer readable storage media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The imaging interface 1006 is configured to receive imaging data from, for example, past CT or MRI scans (or scans received from a similar-sized patient or diagnostic imaging sensors built into the delivery device or template system). The display interface 1008 is configured to transmit display data to a display device 1010. The display device 1010 may be integrated into the guidance system 1000. Alternatively, the display device may be on an existing computing device, such as a smart phone.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

1. A temporary vessel occlusion device, the occlusion device comprising:
a guidewire comprising a distal end and at least one location sensor disposed proximate the distal end, wherein the location sensor is configured to communicate with an external receiver;
a catheter comprising a lumen in fluid communication with at least one access port; and
a temporary embolic agent;
wherein the catheter is configured to be inserted over the guidewire, and to deploy the temporary embolic agent into the vessel.

2. The temporary vessel occlusion device according to claim 1, wherein the temporary embolic agent is selected from group consisting of: degradable starch microspheres, urea and sugar, poly(lactic-co-glycolic acid), PLGA-Polyethylene Glycol-PLGA, carboxymethylcellulose, chitin, hydroxyethyl acrylate, albumin, gelatin, pluronic F127, polyvinyl alcohol, cellulose acetate (CA), polylactic acid (PLA), poly(glycolic acid) (PGA), copolymers of the PLA and PGA, polycaprolactone (PCL), polycaprolactone (PCL), poly(L-lactide-co-ε-caprolactone) (PLCL), and poly(D,L-lactide-co-glycolide) (PLGA).

3. The temporary vessel occlusion device according to claim 2, wherein the temporary embolic agent degrades within the blood vessel within 20 minutes to two hours.

4. The temporary vessel occlusion device according to claim 1, wherein the catheter further comprises at least one communication sensor located at a distal end.

5. The temporary vessel occlusion device according to claim 1, wherein the temporary embolic agent is contained within a biodegradable casing.

6. The temporary vessel occlusion device according to claim 5, wherein the biodegradable casing comprises a starch wafer.

7. The temporary vessel occlusion device according to claim 5, wherein the biodegradable casing comprises a self-expanding scaffold stent.

8. The temporary vessel occlusion device according to claim 1, wherein the catheter further comprises a handle having an automatic deployment mechanism to deploy the temporary embolic agent.

9. The temporary vessel occlusion device according to claim 8, wherein the automatic deployment mechanism comprises a CO2 cartridge.

10. The temporary vessel occlusion device according to claim 1, wherein the location sensor is a NFC sensor.

11. The temporary vessel occlusion device according to claim 1, wherein the catheter further comprises at least one monitoring sensor located at a distal end.

12. The temporary vessel occlusion device according to claim 11, wherein the at least one monitoring sensor is selected from the group consisting of: pressure monitor sensor, pH sensor, oxygen sensor, and CO2 sensor.

13. A kit for temporary vessel occlusion, the kit comprising:
an insertion assisting device comprising at least one communication receiver;
a guidewire comprising at least one location sensor disposed proximate to a distal end of the guidewire and in operative communication with the communication receiver;
a catheter removably positionable over at least a portion of the guidewire, the catheter comprising a lumen in fluid communication with at least one access port; and
a temporary embolic agent;
wherein the communication receiver displays a location of the guidewire within the vessel and the catheter is configured to carry and deploy the temporary embolic agent into the vessel.

14. A kit according to claim 13, wherein the catheter further comprises at least one communication sensor located at a distal end of the catheter.

15. A kit according to claim 13, wherein the catheter further comprises a handle having an automatic deployment mechanism to deploy the temporary embolic agent.

16. The temporary vessel occlusion device according to claim 13, wherein the location sensor comprises a NFC sensor.

17. The temporary vessel occlusion device according to claim 13, wherein the temporary embolic agent degrades within the vessel in a time period of 20 minutes to two hours.

18. A method of providing a temporary occlusion agent to a vessel of a patient, the method comprising:
inserting a guidewire having at least one location sensor into the vessel;
providing an external communication receiver configured to communicate with the location sensor to determine a location of the guidewire in the vessel;
inserting a catheter along at least a portion of the guidewire;
deploying a temporary embolic agent within the vessel; and
removing the guidewire and catheter from the vessel.

19. The method according to claim 18, wherein the catheter further comprises a handle having an deployment mechanism to deploy the temporary embolic agent into the vessel.

20. The method according to claim 18, wherein the communication sensor is a NFC sensor and the external communication receiver is a NFC receiver.
